(19) United States
(12) Patent Application Publication
(10) Pub. No.: US 2020/0078242 A1
(43) Pub. Date: Mar. 12, 2020

(54) THERAPEUTIC MATTRESS

(71) Applicant: Lamont Smith, Erlanger, KY (US)

(72) Inventor: LAMONT SMITH, ERLANGER, KY (US)

(21) Appl. No.: 16/127,127

(22) Filed: Sep. 10, 2018

(51) Int. Cl. A61G 7/07; A47G 9/10; A61B 5/00; A61B 5/021; A61B 5/024

(52) U.S. Cl. CPC A61G 7/072; A47G 9/1036; A61B 5/6892; A61B 5/021; A61B 5/024; A61B 5/4812

(57) ABSTRACT

The enclosed invention provides a therapeutic mattress having at least one aperture therein and a head-sized insert that is placeable within the aperture. More specifically, the head sized insert provides at least one and preferably multiple benefits to a user that lays on the mattress in either a face down or face up position. In practice, the therapeutic life aids a user to achieve restfulness during normal rest and during recovery from sickness and/or injury.

### FIELD OF THE INVENTION

The enclosed invention provides a therapeutic mattress having at least one aperture therein and a head-sized insert that is placeable within the aperture. More specifically, the head sized insert provides at least one and preferably multiple benefits to a user that lays on the mattress in either a face down or face up position.

### BACKGROUND OF THE INVENTION

Since the dawn of time, mankind has sought comfort when he rests. Pre-civilization, humans have used leaves, straw, cotton and more as padding on which to lay and seek rest. In ancient civilizations, mankind created stuffed pads and mattresses to provide much sought after sweet rest.

In more modern times, mattresses of nearly every conceivable configuration have been created to meet the needs of different sleeping types and even to help injured and/or sick persons to better recover. Such mattresses, especially in developed locales, are made from natural materials, man-made materials and composites thereof. Some modern mattresses have sensors that seemingly sense or hear when a user is snoring so as to change the elevation thereof to end a sleeper's snoring.

As technology advances, mattresses are sought that will address ever increasing issues that deter restfulness. These mattresses will serve issues related to restfulness and also help to aid in the recovery processes for the sick and injured.

What is therefore needed is a mattress that provides at least one and preferably multiple benefits to a user by use of an insert into an aperture. The insert should deliver useful benefits to a sleeper or one seeking to recover from injury or illness.

### SUMMARY OF THE INVENTION

Accordingly, the invention provides a therapeutic mattress comprising a first layer, a second layer positioned opposite to the first layer, and a middle layer positioned between the first layer and the second layer. Further, an aperture is positioned onto the first layer. The aperture has a circumference and extends through the first layer, and preferably extends at least partially through the middle layer of the mattress. A face pad insertable within the aperture upon which a user's head or face can rest will laying on the therapeutic mattress.

In one preferred embodiment herein, the aperture extends fully through the middle layer. In another preferred embodiment, the aperture extends at least partially through the second layer. In yet another embodiment herein, the aperture extends fully through the second layer.

The face pad herein is configured to fit the face of a human head. Preferably, the face pad is sizable for differently sized faces. The face pad contains soft padding. It may also provide a heating feature wherein for heating a face or head applied to it. The face pad may also provide a cooling feature wherein for cooling a face or head applied to it. Also preferably, the face pad may provide a vibration feature that may cause the face pad to vibrate against the head or face of a user.

In one embodiment herein, the face pad comprises one or more biometric sensors. One biometric sensor herein tracks a user's sleeping pattern. Another biometric sensor may be used for tracking a user's blood pressure. Yet another biometric sensor tracks a user's heart rate.

The mattress preferably comprises a power source that powers the face pad. In practice, the face pad, once inserted into the aperture, connects to an electrical contact from which electricity flows to the face pad. If the face pad herein comprises one or more electrical elements (e.g., one or more biometric sensors), the connected face pad then becomes powered to function.

When the mattress herein comprises a power source and is electrically powered, it may also be connected to local area network. If so connected, it uses that connection to transmit data from the mattress and/or the face pad to a computer, a server, a cell phone or any sort of application placed upon any of the foregoing. In similar fashion, therapeutic mattress herein provides at least one BLUETOOTH® connection to one or more BLUETOOTH® connectable devices like cell phones and pad devices upon which one or more computer applications exist to receive, interpret and show received data from the mattress and/or face pad.

### BRIEF DESCRIPTION OF THE FIGURES

The various exemplary embodiments of the present invention, which will become more apparent as the description proceeds, are described in the following detailed description in conjunction with the accompanying drawings, in which:

FIG. 1 is a planar view of a user lying face down on the therapeutic mattress;

FIG. 2 is planar view of a user lying face up on the therapeutic mattress;

FIG. 3 is a side view of a user lying face down into the therapeutic mattress herein; and

FIG. 4 is planar view of the aperture of the therapeutic mattress with the face pad inserted therein.

### DETAILED DESCRIPTION OF THE INVENTION

Accordingly, the invention provides a therapeutic mattress comprising a first layer, a second layer positioned opposite to the first layer, and a middle layer positioned between the first layer and the second layer. Further, an aperture is positioned onto the first layer. The aperture has a circumference and extends through the first layer, and preferably extends at least partially through the middle layer of the mattress. A face pad insertable within the aperture upon which a user's head or face can rest will laying on the therapeutic mattress.

The therapeutic mattress or mattress herein is preferably a of the conventional type with respect to materials, structure and support. This is true, particularly, for the non-apertured portions of the mattress. The first layer of the mattress upon which a user lays is pliable yet firm and soft to the touch. In all respects except for the aperture discussed at length herein, the therapeutic mattress is a normal mattress that a user might purchase in any retail outlet that regularly sells them.

In one preferred embodiment herein, the aperture extends fully through the middle layer. In another preferred embodiment, the aperture extends at least partially through the second layer. In yet another embodiment herein, the aperture extends fully through the second layer.

The face pad herein is configured to fit the face of a human head. Preferably, the face pad is sizable for differently sized faces. The face pad contains soft padding. It may also provide a heating feature wherein for heating a face or head applied to it. The face pad may also provide a cooling feature wherein for cooling a face or head applied to it. Also preferably, the face pad may provide a vibration feature that may cause the face pad to vibrate against the head or face of a user.

The face pad or pad herein is also preferably soft to the touch, but firm enough to properly support a user's head and/or neck. The top or covering surface of the pad is both preferably hypoallergenic and/or absorbent for moisture, sweat and oil. The covering surface may comprise cotton, any of a number of man made materials, top sheet material similar to that used in absorbent articles like diapers, satin, silk and blends thereof.

In one embodiment herein, the face pad comprises one or more biometric sensors. One biometric sensor herein tracks a user's sleeping pattern. Another biometric sensor may be used for tracking a user's blood pressure. Yet another biometric sensor tracks a user's heart rate. Persons of skill in the art are well acquainted with the design and use of such sensors in smart wear clothing and watches which are well known.

The mattress preferably comprises a power source that powers the face pad. In practice, the face pad, once inserted into the aperture, connects to an electrical contact from which electricity flows to the face pad. If the face pad herein comprises one or more electrical elements (e.g., one or more biometric sensors), the connected face pad then becomes powered to function.

When the mattress herein comprises a power source and is electrically powered, it may also be connected to local area network. If so connected, it uses that connection to transmit data from the mattress and/or the face pad to a computer, a server, a cell phone or any sort of application placed upon any of the foregoing. In similar fashion, therapeutic mattress herein provides at least one BLUETOOTH® connection to one or more BLUETOOTH® connectable devices like cell phones and pad devices upon which one or more computer applications exist to receive, interpret and show received data from the mattress and/or face pad.

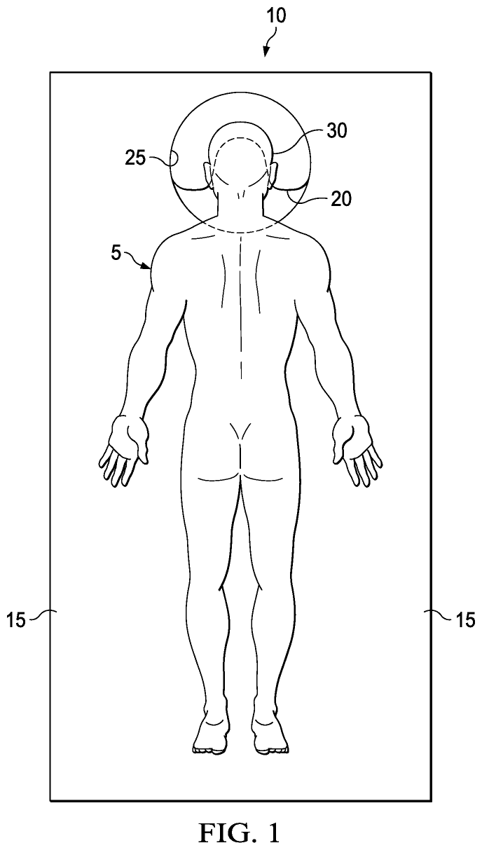

FIG. 1 is a planar view of a user lying face down on the therapeutic mattress. It shows user 5 lying face down onto therapeutic mattress (i.e., “mattress”) 10. Mattress 10 comprises aperture 25 which is an opening into which face pad (i.e., or “pad”) 20 resides. More specifically, pad 20 is configured to sit snugly within, yet removably, from aperture 25. A user's head 30 (and face) lay upon pad 20 for the purpose of resting thereon.

In practice, a user lays face down onto mattress 10 and pad 20. Aperture 25 allows user 5 to draw and expel breath either through openings 27 below aperture 25 and/or through middle layer 18 and second layer 16. Pad 20 allows head 30 to rest comfortably thereon by providing padding thereto. In fact, a preferred embodiment of pad 20 is one in which it is configurable to differently sized heads and faces. This is accomplished through some combination to pad 20 whereby its surface area is at least partially inflatable, fully inflatable, or otherwise mechanically alterable.

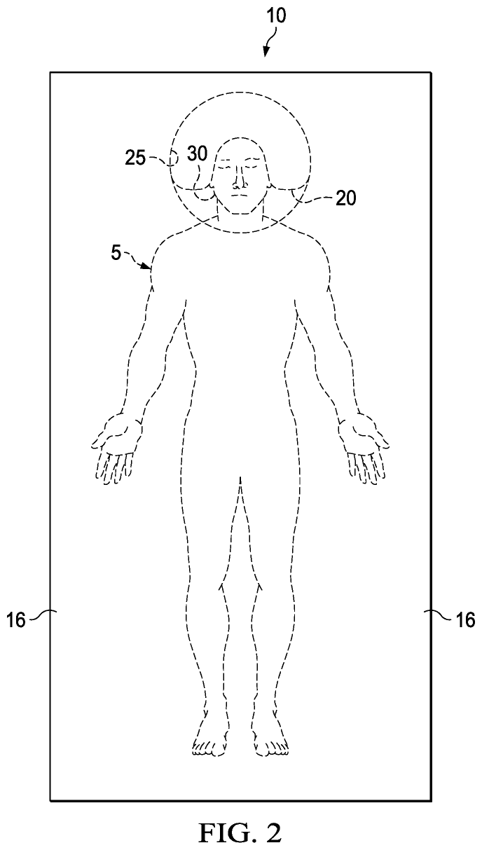

FIG. 2 is a planar view of user 5 lying face up on therapeutic mattress 10. In this rendition, user 5's head rests upon face pad 20 while his face is pointed up, as shown. In this position, pad 20 is able to support user's head while also giving tangential support to his neck. Face pad 20 in FIGS. 1 and 2 is shown in a half crescent configuration with the middle portion open. In another embodiment herein, pad 20 may be configured into a half crescent without the middle portion thereof providing an opening, i.e., a solid half crescent shape. The solid half-crescent shape of pad 20 is configured to provide user 5's head and neck full support when laying thereon.

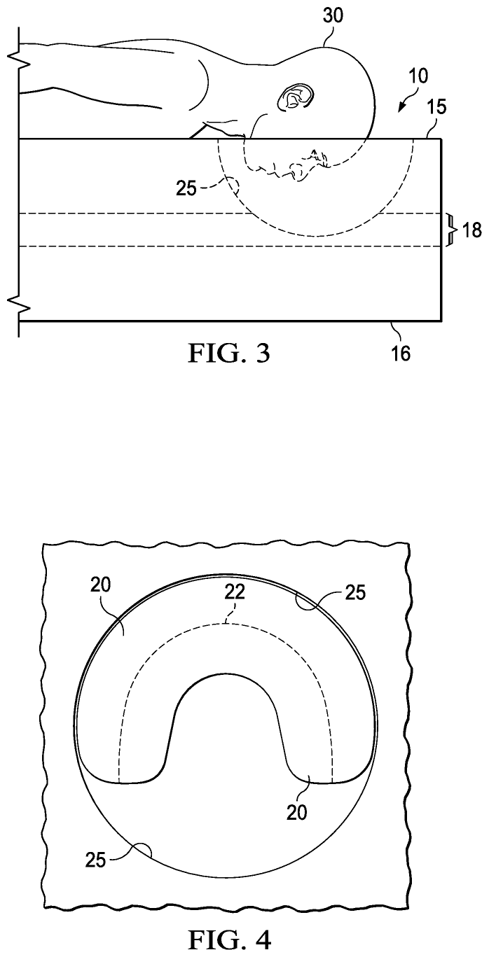

FIG. 3 provides a side view of user 5 lying face down into the therapeutic mattress 10 herein. User 5 is shown lying face down into mattress 10. User 5's head 30 is shown partially submerged within aperture 25. Aperture 25 is shown fully penetrating first layer 15 and partially penetrating middle layer 18. It also possible that aperture 25 could partially or fully penetrate through second layer 16 depending upon manufacturer's design preference.

As shown, opening 27 exists and allows the receipt and expulsion of air in user 5's downward facing position. Air may also be taken in through second layer 16 and middle layer 18. In alternative embodiment, aperture 25 may be designed to cause air to be expelled and received only through middle layer 18 and second layer 16. In that embodiment, such layers may provide filters through air is absorbed through that clean air flowing therethrough. This embodiment would be particularly beneficial for injured or sick users 5 laying upon mattress 10 for rest recovery purposes.

FIG. 4 is planar view of aperture 25 of therapeutic mattress 10 with face pad 20 inserted therein. As shown, face pad 20 fits snugly within aperture 25. Also as shown, pad 20 can be shown by dotted line 22 which is meant to portray a differently sized pad 20. As previously discussed herein, pad 20 may be expanded or decreased to custom fit the head and/or face of user 5. This is an important feature and enables a one-size fits all configuration for pad 20. As also noted, pad 20 may be expanded either by inflation and/or mechanically. If mechanically expanded, pad 20 comprises structure therein that is movable outwardly and inwardly from its center to the outer edges of pad 20.

As noted extensively herein, therapeutic mattress 10 comprises aperture 25. If user 5 desires to use mattress 10 like a regular non-apertured mattress, he may place an insert (not shown) into aperture 25. The insert is configured to fully fill aperture 25 such that once used, first layer 15 of mattress 10 appears and feels like one continued surface upon which user 5 may lay and rest.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A mattress, comprising:
a. A first layer;
b. A second layer positioned opposite to said first layer;
c. A middle layer positioned between said first layer and said second layer;
d. An aperture positioned onto said first layer, said aperture having a circumference and said aperture extending through said first layer and extending at least partially through said middle layer of said mattress; and
e. A face pad insertable within said aperture.

2. The mattress of claim 1 wherein said aperture extends fully through said middle layer.

3. The mattress of claim 2 wherein said aperture extends at least partially through said second layer.

4. The mattress of claim 3 wherein said aperture extends fully through said second layer.

5. The mattress of claim 1 wherein said face pad is configured to fit the face of a human head.

6. The mattress of claim 5 wherein said face pad is sizable for differently sized faces.

7. The mattress of claim 1 wherein said face pad contains soft padding.

8. The mattress of claim 1 wherein said face pad provides a heating feature wherein said face pad may be heated.

9. The mattress of claim 1 wherein said face pad provides a cooling feature wherein said face pad may be cooled.

10. The mattress of claim 1 wherein said face pad provides a vibration feature wherein said face pad can be caused to vibrate.

11. The mattress of claim 1 wherein said face pad comprises one or more biometric sensors.

12. The mattress of claim 11 wherein said face pad comprises a biometric sensor for tracking a user's sleeping pattern.

13. The mattress of claim 11 wherein said face pad comprises a biometric sensor for tracking a user's blood pressure.

14. The mattress of claim 11 wherein said face pad comprises a biometric sensor for tracking a user's heart rate.

15. The mattress of claim 1 wherein said mattress comprises a power source.

16. The mattress of claim 1 wherein said mattress is connectable to a local area network.

17. The mattress of claim wherein said mattress provides at least one BLUETOOTH® connection.
